(19) United States
(12) Patent Application Publication
(10) Pub. No.: US 2023/0384231 A1
(43) Pub. Date: Nov. 30, 2023

(54) METHOD AND SYSTEM FOR PREDICTING SEVERITY OF BACTERIAL BLIGHT OF RICE BASED ON MULTI-PHENOTYPIC PARAMETERS

(71) Applicant: Zhejiang University, Hangzhou City (CN)

(72) Inventors: Yong He, Hangzhou City (CN); Xiulin Bai, Hangzhou City (CN); Xuping Feng, Hangzhou City (CN)

(21) Appl. No.: 18/166,841

(22) Filed: Feb. 9, 2023

(30) Foreign Application Priority Data
May 26, 2022 (CN) 202210587857.2

(51) Int. Cl. G01N 21/84; G01N 21/55; G06Q 10/04; G06Q 10/0639; G06V 20/68

(52) U.S. Cl. CPC G01N 21/84; G01N 21/55; G06Q 10/04; G06Q 10/06393; G06V 20/68; G01N 2021/8466

(57) ABSTRACT

The present disclosure relates to a method and system for predicting a severity of bacterial blight of rice based on multi-phenotypic parameters. The method includes: calculating a rice spectral reflectance corresponding to each of the plots in a study area based on multi-spectral images, and screening characteristic variables based on a rice leaf chlorophyll content and a rice plant water content (WC) under stress of the bacterial blight to establish a new spectral index (SI); predicting the rice leaf chlorophyll content and the rice plant WC in the study area based on the rice spectral reflectance and regression model prediction, and predicting the incidence of the bacterial blight of rice; and obtaining quick indication of the severity of the bacterial blight of rice based on the new SI and the prediction. The method is suitable for high-throughput rice disease phenotype monitoring research.

### CROSS REFERENCE TO RELATED APPLICATION

This patent application claims the benefit and priority of Chinese Patent Application No. 202210587857.2, filed with the China National Intellectual Property Administration on May 26, 2022, the disclosure of which is incorporated by reference herein in its entirety as part of the present application.

### FIELD

The present disclosure relates to the technical field of prediction of a severity of bacterial blight of rice, and in particular, to a method and system for predicting a severity of bacterial blight of rice based on multi-phenotypic parameters.

### BACKGROUND

Rice is one of the main food crops, and disease infection is an important factor affecting rice yield. It is of great significance to discover and understand the incidence of rice diseases in time for plant protection and field management. The traditional disease incidence detection methods are mostly manual, which is affected by human subjective judgment and environmental changes, especially the detection of the incidence of rice in the field requires a lot of manpower, material resources and time. With the rapid development of modern agricultural technology, the unmanned aerial vehicle (UAV) remote sensing technology provides support for rapid monitoring of crop phenotypes in the field. The UAV is equipped with a red-green-blue (RGB) camera, a multi-spectral camera, or a hyperspectral camera to invert crop canopy information, such as chlorophyll content, water content (WC), and nitrogen content. However, the models established by different phenotypic parameters inversion are different, and the data characteristics used are also different. It is rarely reported to use the same data characteristics to invert multiple phenotypic parameters.

The bacterial blight is one of the three major diseases of rice, which seriously affects the yield and quality of rice. The bacterial blight is a bacterial disease caused by the infection of Xanthomonas oryzae pv. Oryzae (Xoo for short). The Xoo proliferates in a large number in the vascular bundle after invading through rice wounds or stomas, leading to blockage of the vascular bundle, and hindering the transportation of nutrients and water in the plant. As a result, the photosynthesis is weakened, the leaf pigment content is reduced, the leaf WC is reduced, and the leaves turn yellow and are withered. Therefore, there is a correlation between the severity of the bacterial blight of rice and the chlorophyll content and WC of rice, but little attention has been paid to this correlation at present.

### SUMMARY

In view of the above problems, the present disclosure provides a method and system for predicting a severity of bacterial blight of rice based on multi-phenotypic parameters, which realizes rapid indication of the severity of the bacterial blight of rice in the field using the multi-spectral remote sensing technology and chlorophyll content and WC changes.

To achieve the above objective, the present disclosure provides the following technical solutions:

A method for predicting a severity of bacterial blight of rice based on multi-phenotypic parameters includes:

obtaining multi-spectral images of rice in a study area, where the study area includes a plurality of plots;

calculating a rice spectral reflectance corresponding to each of the plots based on the multi-spectral images;

determining a rice leaf chlorophyll content corresponding to each of the plots based on the rice spectral reflectance and a first regression model, and determining a rice plant WC corresponding to each of the plots based on the rice spectral reflectance and a second regression model, where the first regression model is determined based on a first sample data set; the second regression model is determined based on a second sample data set; the first sample data set includes a plurality of spectral reflectances of sample rice and a leaf chlorophyll content of the sample rice corresponding to each of the spectral reflectances of the sample rice, and the second sample data set includes the plurality of spectral reflectances of the sample rice and a plant WC of the sample rice corresponding to each of the spectral reflectances of the sample rice; and the sample rice is rice under stress of the bacterial blight;

determining an incidence of the bacterial blight of rice corresponding to each of the plots based on a correlation relationship and the rice leaf chlorophyll content and the rice plant WC corresponding to each of the plots, where the correlation relationship is a correlation relationship between the rice leaf chlorophyll content, the rice plant WC, and the incidence of the bacterial blight of rice;

screening characteristic variables of the rice spectral reflectance based on a score of a variable importance in the projection (VIP) index of the first regression model and a score of a VIP index of the second regression model to obtain a spectral index (SI); and

calculating a correlation between the SI and the incidence of the bacterial blight of rice, and generating a visual distribution map of the severity of the bacterial blight of rice in the study area based on the correlation between the SI and the incidence of the bacterial blight of rice.

Optionally, the method further includes:

calculating a correlation between the SI and the rice leaf chlorophyll content, and generating a visual distribution map of the rice leaf chlorophyll content in the study area based on the correlation between the SI and the rice leaf chlorophyll content;

and/or, calculating a correlation between the SI and the rice plant WC, and generating a visual distribution map of the rice plant WC in the study area based on the correlation between the SI and the rice plant WC;

and/or, calculating the correlation between the SI and the rice leaf chlorophyll content, calculating the correlation between the SI and the rice plant WC, and generating a visual distribution map of the multi-phenotypic parameters of the rice in the study area based on the correlation between the SI and the incidence of the bacterial blight of rice, the correlation between the SI and the rice leaf chlorophyll content, and the correlation between the SI and the rice plant WC, where the multi-phenotypic parameters include the incidence of the bacterial blight of rice, the rice leaf chlorophyll content, and the rice plant WC.

Optionally, the method further includes: constructing a sample database, where the sample database includes the first sample data set, the second sample data set, and a third sample data set; and the third sample data set includes a plurality of incidences of bacterial blight of the sample rice and a leaf chlorophyll content and a plant WC of the sample rice corresponding to each of the incidences of the bacterial blight of the sample rice.

Optionally, a process of determining the leaf chlorophyll content of the sample rice is as follows:

determining the leaf chlorophyll content of the sample rice corresponding to each of the plots in the sample area using a soil-plant analysis development (SPAD)-502 chlorophyll meter.

Optionally, a process of determining the plant WC of the sample rice is as follows:

calculating the plant WC of the sample rice corresponding to each of the plots in the sample area using a wet basis WC method.

Optionally, a process of determining the first regression model is as follows:

determining the first regression model according to a partial least square regression (PLSR) method and the first sample data set.

Optionally, a process of determining the second regression model is as follows:

determining the second regression model according to a PLSR method and the second sample data set.

Optionally, a process of calculating a rice spectral reflectance corresponding to each of the plots based on the multi-spectral images specifically includes:

pre-processing the multi-spectral images; and

extracting rice spectral reflectances from the pre-processed multi-spectral images using environment for visualizing images (ENVI) software, so as to determine the rice spectral reflectance corresponding to each of the plots.

A system for predicting a severity of bacterial blight of rice based on multi-phenotypic parameters includes:

a multi-spectral image obtaining module configured to obtain multi-spectral images of rice in a study area, where the study area includes a plurality of plots;

a rice spectral reflectance calculation module configured to calculate a rice spectral reflectance corresponding to each of the plots based on the multi-spectral images;

a rice leaf chlorophyll content and rice plant WC calculation module configured to determine a rice leaf chlorophyll content corresponding to each of the plots based on the rice spectral reflectance and a first regression model, and determine a rice plant WC corresponding to each of the plots based on the rice spectral reflectance and a second regression model, where the first regression model is determined based on a first sample data set; the second regression model is determined based on a second sample data set; the first sample data set includes a plurality of spectral reflectances of sample rice and a leaf chlorophyll content of the sample rice corresponding to each of the spectral reflectances of the sample rice, and the second sample data set includes the plurality of spectral reflectances of the sample rice and a plant WC of the sample rice corresponding to each of the spectral reflectances of the sample rice; and the sample rice is rice under stress of the bacterial blight;

a rice bacterial blight incidence determination module configured to determine an incidence of the bacterial blight of rice corresponding to each of the plots based on a correlation relationship and the rice leaf chlorophyll content and the rice plant WC corresponding to each of the plots, where the correlation relationship is a correlation relationship between the rice leaf chlorophyll content, the rice plant WC, and the incidence of the bacterial blight of rice;

an SI determination module configured to screen characteristic variables of the rice spectral reflectance based on a score of a VIP index of the first regression model and a score of a VIP index of the second regression model to obtain an SI; and

a visual distribution map generation module for the severity of the bacterial blight of rice configured to calculate a correlation between the SI and the incidence of the bacterial blight of rice, and generate a visual distribution map of the severity of the bacterial blight of rice in the study area based on the correlation between the SI and the incidence of the bacterial blight of rice.

Optionally, the system further includes:

a visual distribution map generation module for the rice leaf chlorophyll content configured to calculate a correlation between the SI and the rice leaf chlorophyll content, and generate a visual distribution map of the rice leaf chlorophyll content in the study area based on the correlation between the SI and the rice leaf chlorophyll content;

and/or, a visual distribution map generation module for the rice plant WC configured to calculate a correlation between the SI and the rice plant WC, and generate a visual distribution map of the rice plant WC in the study area based on the correlation between the SI and the rice plant WC;

and/or, a visual distribution map generation module for the multi-phenotypic parameters of the rice configured to calculate the correlation between the SI and the rice leaf chlorophyll content, calculate the correlation between the SI and the rice plant WC, and generate a visual distribution map of the multi-phenotypic parameters of the rice in the study area based on the correlation between the SI and the incidence of the bacterial blight of rice, the correlation between the SI and the rice leaf chlorophyll content, and the correlation between the SI and the rice plant WC, where the multi-phenotypic parameters include the incidence of the bacterial blight of rice, the rice leaf chlorophyll content, and the rice plant WC.

According to the specific examples provided by the present disclosure, the present disclosure discloses the following technical effects:

(1) Characteristic variables are screened based on a rice leaf chlorophyll content and a rice plant WC under stress of the bacterial blight to establish a new SI. (2) The rice leaf chlorophyll content and the rice plant WC in the study area are predicted based on the rice spectral reflectance and regression model prediction, and the incidence of the bacterial blight of rice is predicted. (3) Quick indication of the severity of the bacterial blight of rice is obtained based on the new SI and the prediction. The method is suitable for high-throughput rice disease phenotype monitoring research.

### BRIEF DESCRIPTION OF THE DRAWINGS

To describe the examples of the present disclosure or the technical solutions in the related art more clearly, the accompanying drawings required in the examples are briefly introduced below. Obviously, the accompanying drawings described below are only some examples of the present disclosure. Those of ordinary skill in the art may further obtain other accompanying drawings based on these accompanying drawings without creative labor.

FIG. 1 is a flow chart of a method for predicting a severity of bacterial blight of rice based on multi-phenotypic parameters of the present disclosure;

FIG. 2 is a flow diagram of an overall implementation mode of the method for predicting a severity of bacterial blight of rice based on multi-phenotypic parameters of the present disclosure;

FIG. 3A shows a relationship change diagram of an SPAD value with a disease severity of rice measured in a field in the present disclosure;

FIG. 3B shows a relationship change diagram of a WC with a disease severity of rice measured in a field in the present disclosure;

FIG. 4 shows Pearson correlation analysis result diagrams of the severity of bacterial blight, the SPAD value, and the WC of rice in the field in the present disclosure;

FIG. 5A is a PLSR model result diagram of the present disclosure;

FIG. 5B is a PLSR model result diagram of the present disclosure;

FIG. 6 is a result diagram of a VIP score of the present disclosure;

FIG. 7 is a scatter diagram between an established SI and the severity of the bacterial blight of rice in the field in the present disclosure;

FIG. 8 is a scatter diagram between the established SI and the SPAD value of rice in the field in the present disclosure;

FIG. 9 is a scatter diagram between the established SI and the WC of rice in the field in the present disclosure;

FIG. 10 is a visual distribution map of the severity of the bacterial blight of rice in the field in the present disclosure; and

FIG. 11 is a result diagram of a system for predicting a severity of bacterial blight of rice based on multi-phenotypic parameters of the present disclosure.

### DETAILED DESCRIPTION

The technical solutions of the examples of the present disclosure are clearly and completely described below with reference to the accompanying drawings. Apparently, the described examples are merely a part rather than all of the examples of the present disclosure. All other examples obtained by those of ordinary skill in the art based on the examples of the present disclosure without creative efforts shall fall within the protection scope of the present disclosure.

To make the above-mentioned objective, features, and advantages of the present disclosure clearer and more comprehensible, the present disclosure will be further described in detail below in conjunction with the accompanying drawings and specific implementations.

### Example I

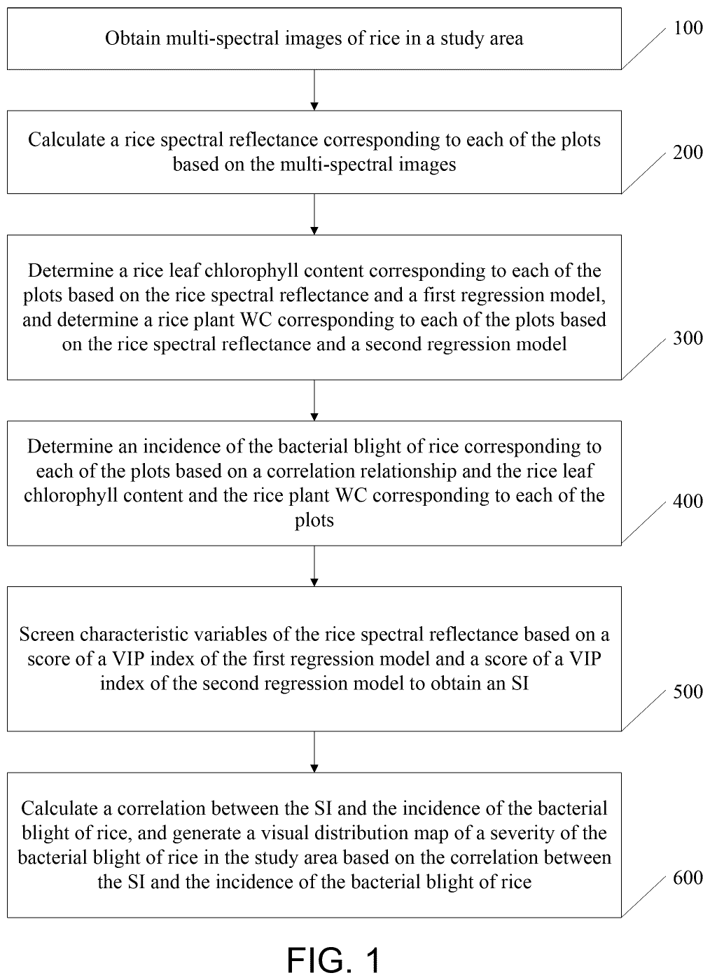

As shown in FIG. 1, a method for predicting a severity of bacterial blight of rice based on multi-phenotypic parameters provided by this example includes the following steps.

Step 100: Multi-spectral images of rice in a study area were obtained. The study area included a plurality of plots.

Step 200: A rice spectral reflectance corresponding to each of the plots was calculated based on the multi-spectral images.

Step 300: A rice leaf chlorophyll content corresponding to each of the plots was determined based on the rice spectral reflectance and a first regression model, and a rice plant WC corresponding to each of the plots was determined based on the rice spectral reflectance and a second regression model. The first regression model was determined based on a first sample data set. The second regression model was determined based on a second sample data set. The first sample data set included a plurality of spectral reflectances of sample rice and a leaf chlorophyll content of the sample rice corresponding to each of the spectral reflectances of the sample rice, and the second sample data set included the plurality of spectral reflectances of the sample rice and a plant WC of the sample rice corresponding to each of the spectral reflectances of the sample rice. The sample rice was rice under stress of the bacterial blight.

Step 400: An incidence of the bacterial blight of rice corresponding to each of the plots was determined based on a correlation relationship and the rice leaf chlorophyll content and the rice plant WC corresponding to each of the plots. The correlation relationship was a correlation relationship between the rice leaf chlorophyll content, the rice plant WC, and the incidence of the bacterial blight of rice.

Step 500: Characteristic variables of the rice spectral reflectance were screened based on a score of a VIP index of the first regression model and a score of a VIP index of the second regression model to obtain a SI.

Step 600: A correlation between the SI and the incidence of the bacterial blight of rice was calculated, and a visual distribution map of the severity of the bacterial blight of rice in the study area was generated based on the correlation between the SI and the incidence of the bacterial blight of rice.

Further, the method provided by this example further included the following steps.

A correlation between the SI and the rice leaf chlorophyll content was calculated, and a visual distribution map of the rice leaf chlorophyll content in the study area was generated based on the correlation between the SI and the rice leaf chlorophyll content.

And/or, a correlation between the SI and the rice plant WC was calculated, and a visual distribution map of the rice plant WC in the study area was generated based on the correlation between the SI and the rice plant WC.

And/or, the correlation between the SI and the rice leaf chlorophyll content was calculated, the correlation between the SI and the rice plant WC was calculated, and a visual distribution map of the multi-phenotypic parameters of the rice in the study area was generated based on the correlation between the SI and the incidence of the bacterial blight of rice, the correlation between the SI and the rice leaf chlorophyll content, and the correlation between the SI and the rice plant WC. The multi-phenotypic parameters included the incidence of the bacterial blight of rice, the rice leaf chlorophyll content, and the rice plant WC.

Further, the method provided by this example further included: constructing a sample database. The sample database included the first sample data set, the second sample data set, and a third sample data set. The third sample data set included a plurality of incidences of bacterial blight of the sample rice and a leaf chlorophyll content and a plant WC of the sample rice corresponding to each of the incidences of the bacterial blight of the sample rice.

In the process of constructing the sample database, a process of determining each element was described below.

A process of determining the spectral reflectance of sample rice was as follows.

The multi-spectral images of rice under stress of the bacterial blight in the sample area were obtained using a multi-spectral camera carried by a UAV. Image splicing and background removal were conducted on the multi-spectral images, and the spectral reflectance of the sample rice of each plot in the sample area was extracted based on the processed multi-spectral images and recorded as X.

A process of determining the leaf chlorophyll content of the sample rice was as follows.

The leaf chlorophyll content of the sample rice corresponding to each of the plots under stress of the bacterial blight in the sample area was acquired using an SPAD-502 chlorophyll meter and recorded as Y1.

An example was as follows: the leaf chlorophyll content of the sample rice was obtained using the SPAD-502 chlorophyll meter, and the SPAD value was used to replace the leaf chlorophyll content.

During the measurement, the main leaf vein shall be avoided. Each leaf was regarded as the upper, middle and lower parts from the tip to the sheath. 3 sampling points were randomly selected for each part to measure the SPAD value, and the average SPAD value of the 9 sampling points represented the SPAD value of the rice leaf.

A process of determining the plant WC of the sample rice was as follows.

The plant WC of the sample rice corresponding to each of the plots in the sample area was calculated using a wet basis WC method and recorded as Y2. The calculation formula is as follows:

$$W C = \frac{F W - D W}{F W} \times 100 \%$$

where WC represents the plant WC of the sample rice, FW represents a fresh weight (g) of the sample rice plants, and DW represents a dry weight (g) of the sample rice plants.

A process of determining the incidence of the bacterial blight of the sample rice was as follows.

The investigation on the incidence of the bacterial blight of the sample rice corresponding to each of the plots in the sample area was conducted according to the China national standard GB/T 17980.19-2000. The three plant protection experts conducted field investigation and scored on the day of UAV flight operation, and the average score of the three experts was taken as the final disease severity score, that is, the incidence of the bacterial blight of the sample rice, which was recorded as Y3.

On this basis, the correlation relationship was further determined, that is, according to the third data sample set, the correlation between Y1, Y2, and Y3 was calculated, and the correlation relationship between the rice leaf chlorophyll content, the rice plant WC, and the incidence of the bacterial blight of the rice was obtained.

The correlation was calculated using Pearson correlation analysis, and a correlation coefficient (r) was calculated using SPSS software. r>0 was positive correlation, r<0 was negative correlation, r=0 indicated no linear relationship, |r|=1 indicated completely linear correlation, 0<|r|<=0.3 indicated extremely low correlation, 0.3<|r|<=0.5 indicated low correlation, 0.5<|r|<=0.8 indicated significant correlation, and |r|>0.8 indicated extremely high correlation.

A process of determining the first regression model was as follows.

The first regression model was determined according to a PLSR method and the first sample data set.

A process of determining the second regression model was as follows.

The second regression model was determined according to a PLSR method and the second sample data set.

As a preferred implementation, a process of calculating a rice spectral reflectance corresponding to each of the plots based on the multi-spectral images specifically included the following steps.

The multi-spectral images were pre-processed. Rice spectral reflectances were extracted from the pre-processed multi-spectral images using ENVI software, so as to determine the rice spectral reflectance corresponding to each of the plots.

This example provided a method for predicting a severity of bacterial blight of rice based on multi-phenotypic parameters, mainly including: obtaining the spectral reflectance of rice under the stress of the bacterial blight using the multi-spectral camera carried by the UAV, constructing a regression model between the spectral reflectance and the changes in the chlorophyll content and WC of rice under the stress of the bacterial blight, and selecting corresponding characteristic bands to establish an SI to evaluate the severity of bacterial blight of rice. The SI could be used to quickly indicate the field distribution of the rice leaf chlorophyll content, the rice plant WC, and the incidence of the bacterial blight of rice. Obviously, this example can realize the quick indication of various phenotypic parameters such as the rice leaf chlorophyll content, the rice plant WC, and the incidence of the bacterial blight of rice under the stress of the bacterial blight.

### Example II

First, the method provided in this example was taken as an example to illustrate the feasibility of a method for predicting a severity of bacterial blight of rice based on multi-phenotypic parameters.

The experimental data or sample data of this example involved two experimental sites, namely the rice experimental base in Longyou County, Quzhou City, Zhejiang Province (29° 0′ 17″ N, 119° 10′ 46″ E) and the rice experimental base in Zhuji City, Shaoxing City (29° 37′ 22″ N, 120° 11′ 40″ E). These two sites both belonged to the subtropical monsoon climate, which was humid and rainy and suitable for rice growth, and belonged to the epidemic areas of bacterial blight, which occurred naturally without additional treatment after rice planting. There were 60 plots in the experimental area of the rice experimental base in Longyou County. Each plot was planted with one rice variety, 60 varieties in total, ranging from ASH1 to ASH60. Each plot was 10.6 m long and 4.72 m wide, and the interval between plots was 0.5 m. All the rice was cultivated in mid-May 2021, transplanted to the field manually on June 26, and harvested in mid-October. There were 13 plots in the experimental area of the rice experimental base in Zhuji City. Each plot was 60 m long and 5.5 m wide. Each plot was planted with one rice variety, namely Yongyou 31, Jiaheyou 2, Yong 1578, Zhongzheyou 8, Chunxian 7860, Yongyou 15, Jiaheyou 7245, Yongyou 7860, Yongyou 1540, Jia 67, Zhejing 100, Zhejing 165, and Huaxi 2171. The interval between plots was 0.5 m. All the rice was cultivated in mid-May 2021, transplanted to the field manually on June 13, and harvested in mid-October. All rice varieties were provided by Zhejiang Academy of Agricultural Sciences, and rice growth management was conducted according to local management methods. Different rice varieties had different resistance to bacterial blight, and their phenotypes in the field were also different.

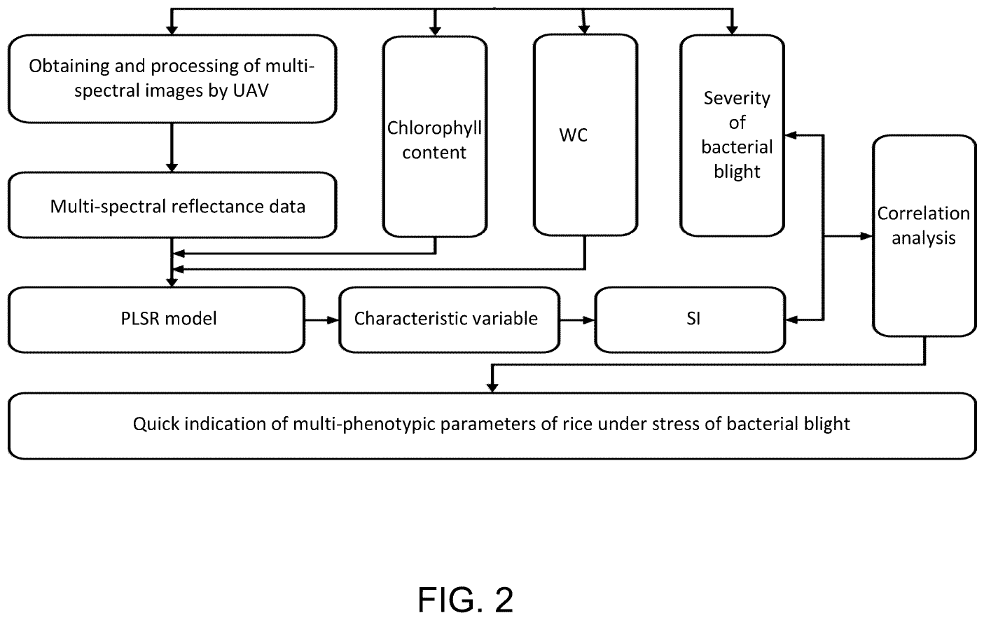

As shown in FIG. 2, the method for predicting a severity of bacterial blight of rice based on changes in chlorophyll and WC provided by this example includes the following steps.

(1) UAV multi-spectral image acquisition was conducted. The multi-spectral images of the experimental base were acquired using the UAV equipped with a 25 band multi-spectral camera, with the wavelength range of 600-875 nm. The multi-spectral images of the rice experimental base in Longyou County were acquired on the 16th, 66th and 92nd days after the rice was transplanted to the field. The multi-spectral images of the rice experimental base in Zhuji City were acquired on the 30th, 68th and 106th days after the rice was transplanted to the field, involving the tillering, jointing, heading and filling stages of rice. During UAV operation, the distance between the UAV and ground was 25 m. The flight speed was 2.5 m/s, the fore-and-aft overlap rate was 60% , and the lateral overlap rate was 75%.

(2) Spectral reflectance (X) extraction was conducted. The multi-spectral images acquired by the UAV were continuous images with a small viewing angle range. The Photoscan software was used to splice these images with a small viewing angle range into the ortho-image of the whole experimental area. The threshold segmentation method was used to remove the background information, and the ENVI software was used to select the plots of interest for spectral reflectance extraction. The threshold segmentation method was based on the wavelength with the largest reflectance difference between the background and rice as the threshold. In this example, the reflectance difference was the largest at the wavelength of 675 nm.

(3) Measurement of the rice chlorophyll content (Y1) was conducted. The rice chlorophyll of each plot in the experimental area was measured using the SPAD-502 chlorophyll meter on the same day of UAV flight operation, and the SPAD value was used to replace the chlorophyll content. Three rice plants were selected randomly in each plot, and three fully expanded leaves were selected randomly for each rice plant. Each leaf was regarded as the upper, middle and lower parts from the tip to the sheath. 3 sampling points were randomly selected for each part to measure the SPAD value, the average SPAD value of the 27 sampling points represented the SPAD value of the rice plant, and the average value of the randomly selected three rice plants represented the SPAD value of the rice in the plot.

(4) Measurement of the rice WC (Y2) was conducted. On the same day of UAV flight operation, three rice plants were selected randomly in each plot in the experimental area. After wiping, the fresh weight was measured and recorded. The rice samples were put in an oven at 105° C. and fixation was conducted for 30 min. The oven was set at 70° C. for drying to constant weight. The sample was weighed, and the dry weight was recorded. The WC of the rice was calculated using a wet basis WC method. The average value of three samples in each plot represented the WC of the plot.

(5) Investigation of the severity of bacterial blight of rice in the field (Y3) was conducted. On the same day of UAV flight operation, according to the China national standard GB/T 17980.19-2000, the three plant protection experts conducted field investigation and scored the disease severity. The disease severity scores were 0, 1, 2, 3, 4, and 5 respectively. 0 meant no obvious disease, 1-5 meant the disease seventies increased in sequence, and finally the average score of the three experts was taken as the final disease severity score result.

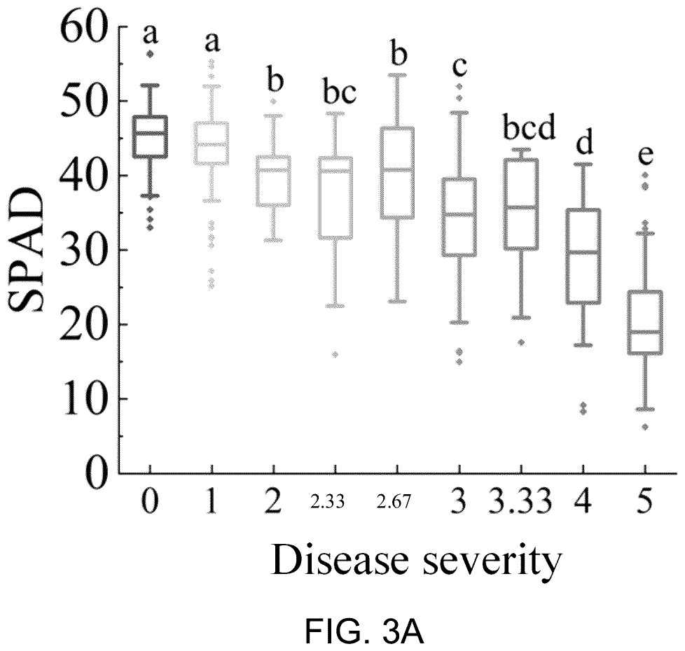

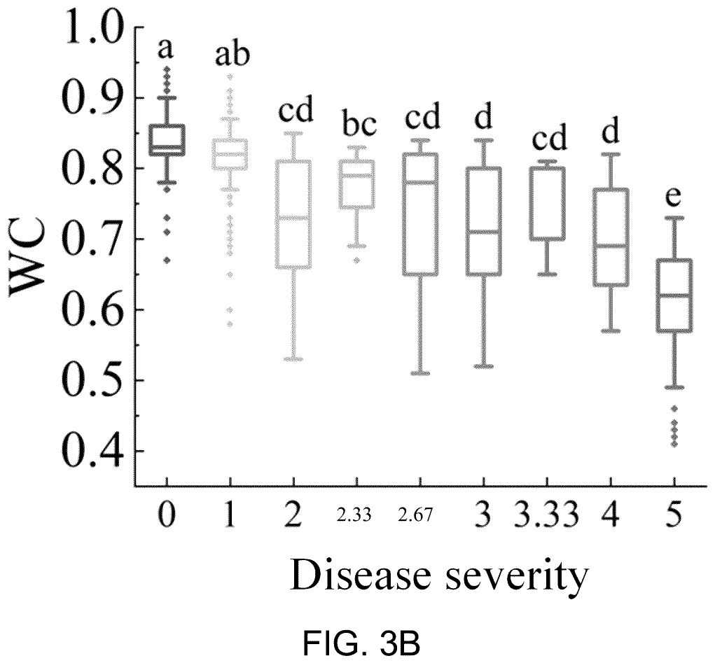

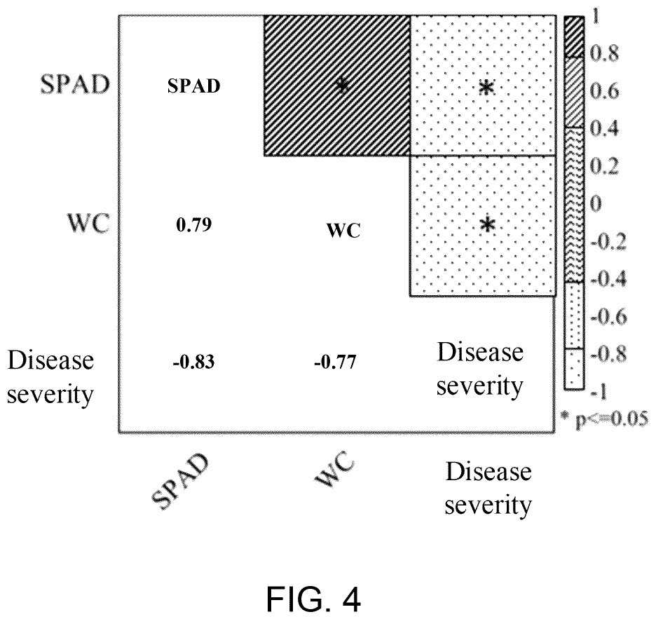

(6) In order to establish a universal model, the data of the two experimental areas was put together for comprehensive analysis. FIG. 3A shows distribution of the SPAD values of rice under different disease seventies, and FIG. 3B shows distribution of the WCs of rice under different disease seventies. Due to the large area of each plot in the experimental area, in order to ensure the reliability of the results, when the spectral data was extracted, each plot in the experimental area of the rice experimental base in Longyou County was divided into two sub plots on average, and each plot in the experimental area of the rice experimental base in Zhuji City was divided into five sub plots on average, so there were 555 samples in total. Pearson correlation analysis was conducted on the SPAD, the WC, and the disease severity of rice. The results are shown in FIG. 4. The disease severity of rice was negatively correlated with the SPAD and the WC of rice, and the correlation coefficients were −0.83 and −0.77 respectively. *p<0.05 indicated that the correlation was significant at 0.05, showing a significant correlation. After one-to-one correspondence of the obtained X, Y1, Y2, and Y3, a modeling set and a prediction set were formed by random selection at a ratio of 8:2. This step also determined the correlation relationship among the rice leaf chlorophyll content, the rice plant WC, and the incidence of the bacterial blight of rice, and the correlation relationship was used in the subsequent practical operation process to determine the incidence of the bacterial blight of rice according to the rice leaf chlorophyll content and the rice plant WC.

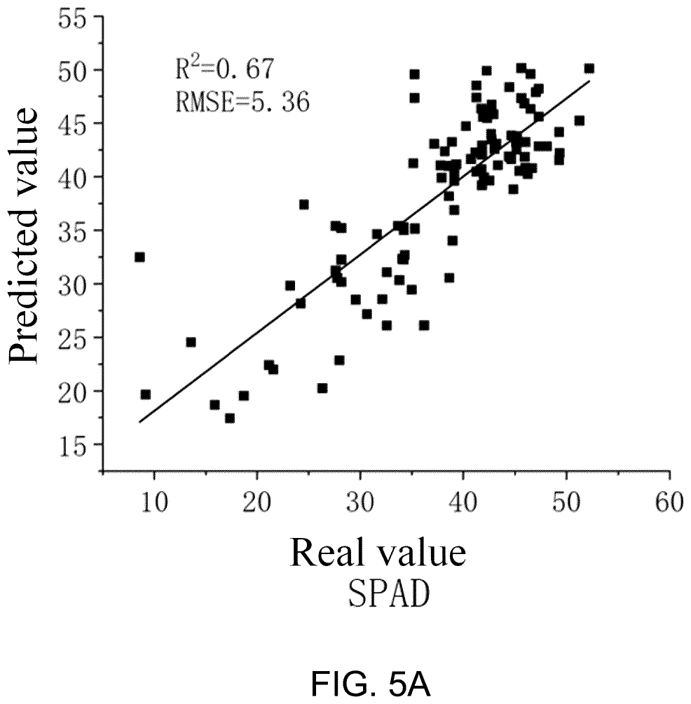

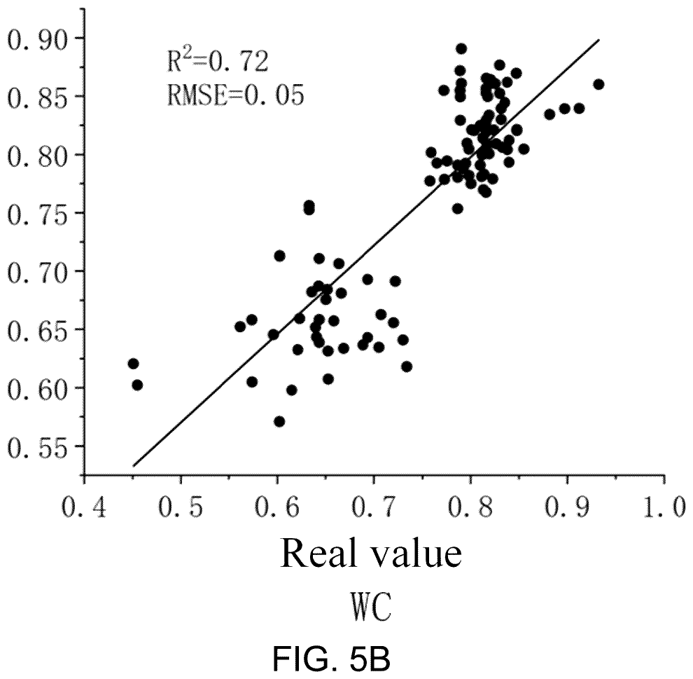

(7) With X in the modeling set as the input and Y1 and Y2 as the output, a PLSR model of the chlorophyll, WC, and spectral reflectance of rice under the stress of the bacterial blight was established, and the corresponding prediction data of the samples in the modeling set were obtained. X in the prediction set was substituted into the model to obtain the prediction data corresponding to the samples in the prediction set. It was feasible to predict SPAD and WC under the stress of the bacterial blight of rice using multi-spectral data for PLSR model result phenotype. For the prediction set of the SPAD, R² is 0.67, and the root mean square error (RMSE) is 5.36, as shown in FIG. 5A. For the prediction set of the WC, R² is 0.72, and the RMSE is 0.05, as shown in FIG. 5B. The PLSR model in this step was used in the subsequent practical operation process to obtain the rice leaf chlorophyll content and the rice plant WC only according to the spectral reflectance.

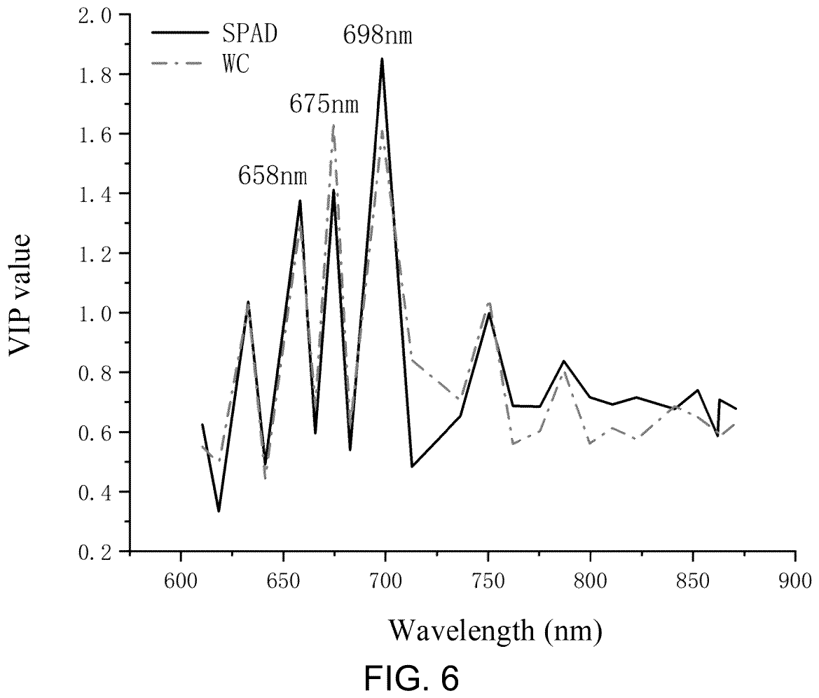

(8) The VIP scores of the PLSR model of the SPAD and the WC of the rice were calculated respectively. The results are shown in FIG. 6. Those with VIP values greater than 1 were regarded as important variables. The spectral data were used to predict the SPAD and the WC of rice respectively. The results indicated that 658 nm, 675 nm and 698 nm were important wavelengths. Based on these three wavelengths, a new SI was established according to whether there was a correlation with the disease severity.

The reflectances of all samples at 658 nm, 675 nm, and 698 nm increased with the disease severity. To reflect the difference between disease seventies, R658+R698 and R658+R675 were calculated respectively, and were added together. Finally, the SI calculation formula was as follows:

SI=2*R658+R675+R698,

where R658, R675, and R698 represented the corresponding spectral reflectances at the wavelengths of 658 nm, 675 nm and 698 nm respectively.

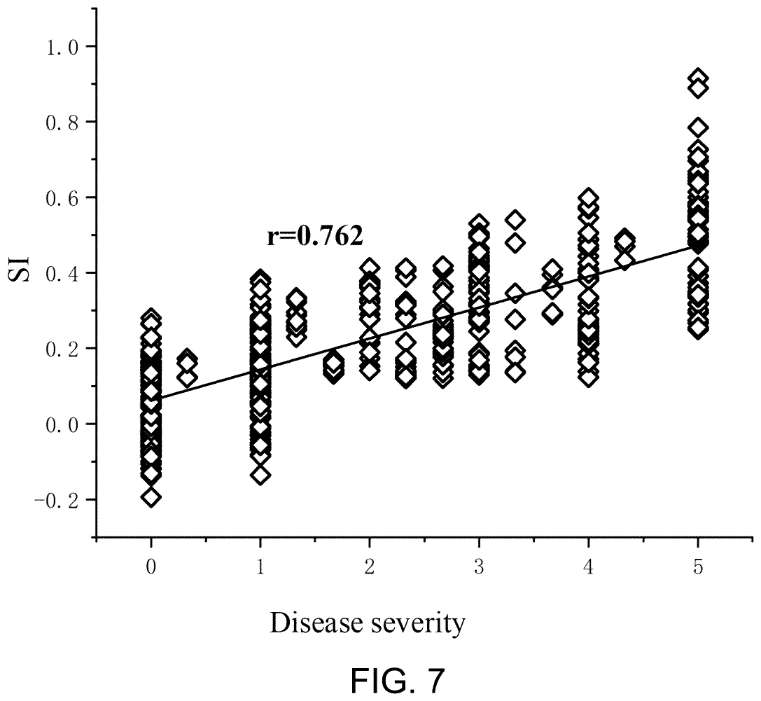

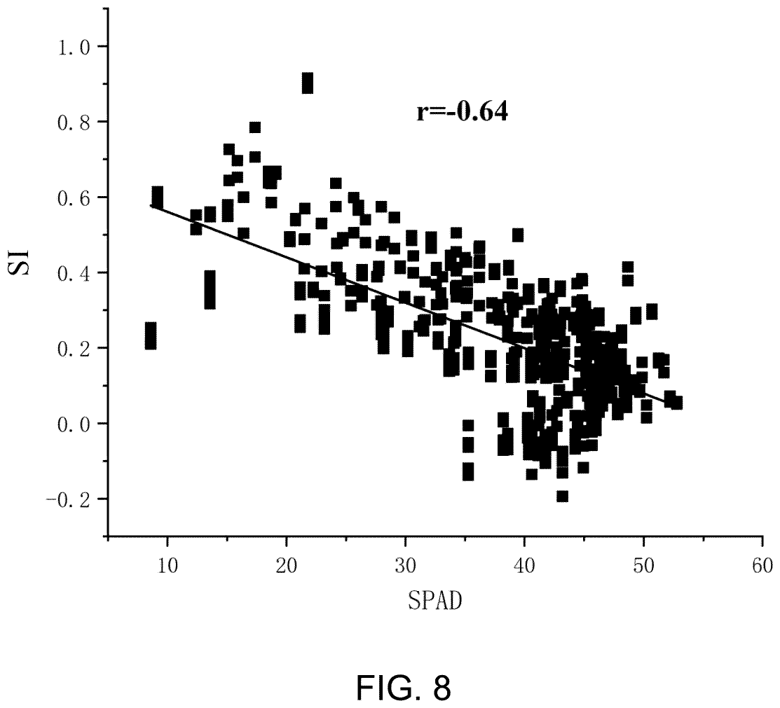

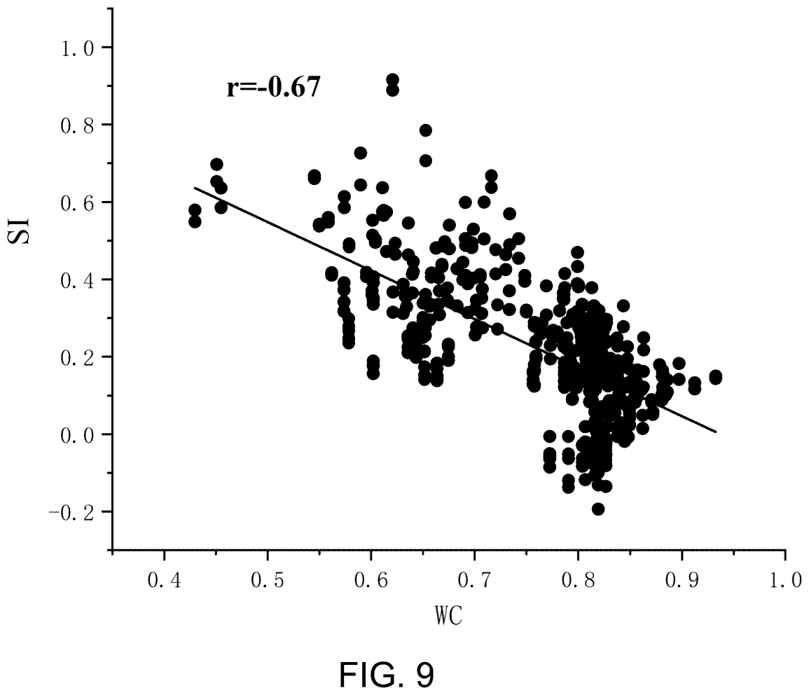

There is a positive correlation between the SI and the disease severity, as shown in FIG. 7, and the correlation coefficient was 0.762, which proved that the SI for evaluation of the severity of bacterial blight of rice established based on the SPAD and WC change of rice was effective. With 0.1 as the threshold, healthy rice and susceptible rice could be quickly distinguished. If SI<=0.1, it was considered as healthy rice without disease, and if SI>0.1, it was considered as susceptible rice. In addition, the correlation between the SI and the rice SPAD as well as the WC is shown in FIG. 8 and FIG. 9, showing a negative correlation and significant correlation. Based on the SI of the corresponding disease severity, the corresponding situation of SPAD and WC could be known.

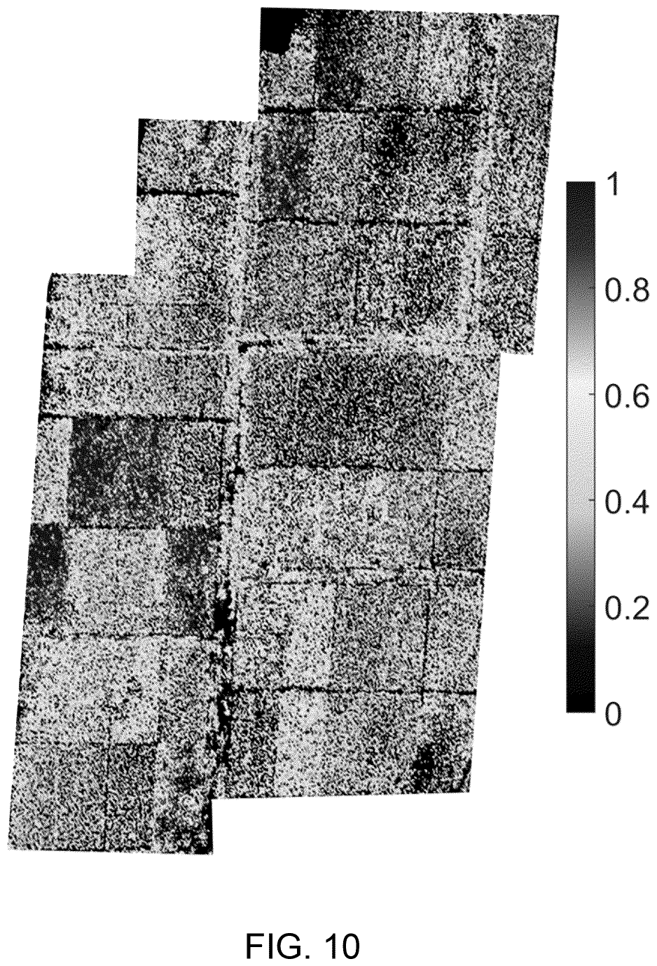

(9) Based on the established SI, the visual analysis of disease severity of rice in the field could be realized, and the SPAD and WC in the field could also be reflected. The visual distribution results are shown in FIG. 10. Due to limited space, only the second flight image of the experimental area in the rice experimental base in Longyou County was selected for display.

### Example III

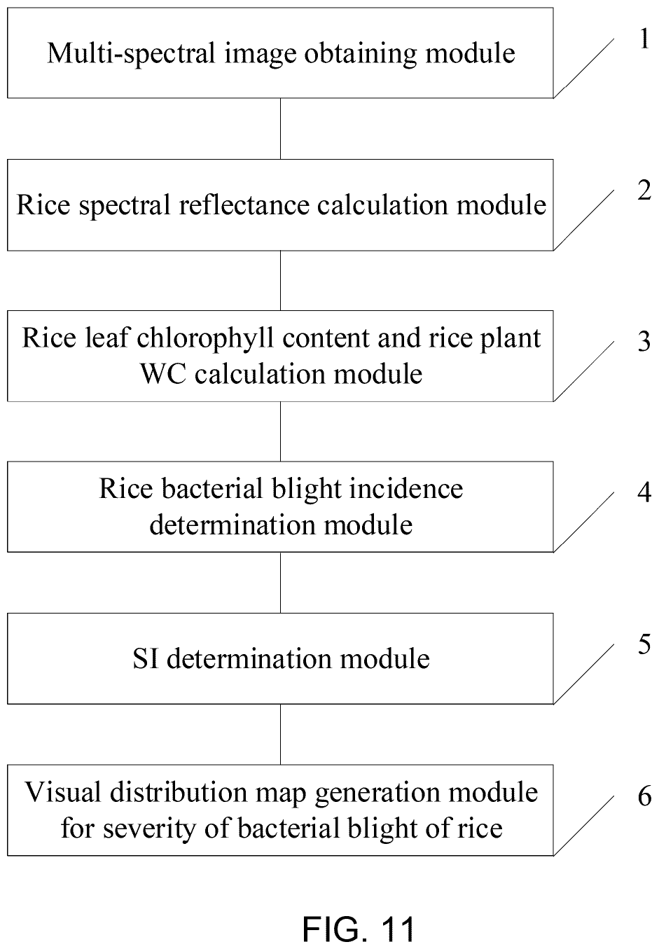

As shown in FIG. 11, a system for predicting a severity of bacterial blight of rice based on multi-phenotypic parameters provided by this examples includes: a multi-spectral image obtaining module 1, a rice spectral reflectance calculation module 2, a rice leaf chlorophyll content and rice plant WC calculation module 3, a rice bacterial blight incidence determination module 4, an SI determination module 5, and a visual distribution map generation module for the severity of the bacterial blight of rice 6.

The multi-spectral image obtaining module 1 was configured to obtain multi-spectral images of rice in a study area. The study area included a plurality of plots.

The rice spectral reflectance calculation module 2 was configured to calculate a rice spectral reflectance corresponding to each of the plots based on the multi-spectral images.

The rice leaf chlorophyll content and rice plant WC calculation module 3 was configured to determine a rice leaf chlorophyll content corresponding to each of the plots based on the rice spectral reflectance and a first regression model, and determine a rice plant WC corresponding to each of the plots based on the rice spectral reflectance and a second regression model. The first regression model was determined based on a first sample data set. The second regression model was determined based on a second sample data set. The first sample data set included a plurality of spectral reflectances of sample rice and a leaf chlorophyll content of the sample rice corresponding to each of the spectral reflectances of the sample rice, and the second sample data set included the plurality of spectral reflectances of the sample rice and a plant WC of the sample rice corresponding to each of the spectral reflectances of the sample rice. The sample rice was rice under stress of the bacterial blight.

The rice bacterial blight incidence determination module 4 was configured to determine an incidence of the bacterial blight of rice corresponding to each of the plots based on a correlation relationship and the rice leaf chlorophyll content and the rice plant WC corresponding to each of the plots. The correlation relationship was a correlation relationship between the rice leaf chlorophyll content, the rice plant WC, and the incidence of the bacterial blight of rice.

The SI determination module 5 was configured to screen characteristic variables of the rice spectral reflectance based on a score of a VIP index of the first regression model and a score of a VIP index of the second regression model to obtain an SI.

The visual distribution map generation module for the severity of the bacterial blight of rice 6 was configured to calculate a correlation between the SI and the incidence of the bacterial blight of rice, and generate a visual distribution map of the severity of the bacterial blight of rice in the study area based on the correlation between the SI and the incidence of the bacterial blight of rice.

Further, the system provided by this examples included: a visual distribution map generation module for the rice leaf chlorophyll content, and/or, a visual distribution map generation module for the rice plant WC, and/or, a visual distribution map generation module for the multi-phenotypic parameters of the rice.

The visual distribution map generation module for the rice leaf chlorophyll content was configured to calculate a correlation between the SI and the rice leaf chlorophyll content, and generate a visual distribution map of the rice leaf chlorophyll content in the study area based on the correlation between the SI and the rice leaf chlorophyll content.

And/or, the visual distribution map generation module for the rice plant WC was configured to calculate a correlation between the SI and the rice plant WC, and generate a visual distribution map of the rice plant WC in the study area based on the correlation between the SI and the rice plant WC. And/or, the visual distribution map generation module for the multi-phenotypic parameters of the rice was configured to calculate the correlation between the SI and the rice leaf chlorophyll content, calculate the correlation between the SI and the rice plant WC, and generate a visual distribution map of the multi-phenotypic parameters of the rice in the study area based on the correlation between the SI and the incidence of the bacterial blight of rice, the correlation between the SI and the rice leaf chlorophyll content, and the correlation between the SI and the rice plant WC. The multi-phenotypic parameters included the incidence of the bacterial blight of rice, the rice leaf chlorophyll content, and the rice plant WC.

Each example of the present specification is described in a progressive manner, each example focuses on the difference from other examples, and the same and similar parts between the examples may refer to each other. Since the system disclosed in an example corresponds to the method disclosed in another example, the description is relatively simple, and reference can be made to the method description.

Specific examples are used herein to explain the principles and implementations of the present disclosure. The foregoing description of the examples is merely intended to help understand the method of the present disclosure and its core ideas; besides, various modifications may be made by those of ordinary skill in the art to specific implementations and the scope of application in accordance with the ideas of the present disclosure. In conclusion, the content of the present specification shall not be construed as limitations to the present disclosure.

1. A method for predicting a severity of bacterial blight of rice based on multi-phenotypic parameters, comprising:
obtaining multi-spectral images of rice in a study area, wherein the study area comprises a plurality of plots;
calculating a rice spectral reflectance corresponding to each of the plots based on the multi-spectral images;
determining a rice leaf chlorophyll content corresponding to each of the plots based on the rice spectral reflectance and a first regression model, and determining a rice plant water content (WC) corresponding to each of the plots based on the rice spectral reflectance and a second regression model, wherein the first regression model is determined based on a first sample data set; the second regression model is determined based on a second sample data set; the first sample data set comprises a plurality of spectral reflectances of sample rice and a leaf chlorophyll content of the sample rice corresponding to each of the spectral reflectances of the sample rice, and the second sample data set comprises the plurality of spectral reflectances of the sample rice and a plant WC of the sample rice corresponding to each of the spectral reflectances of the sample rice; and the sample rice is rice under stress of the bacterial blight;
determining an incidence of the bacterial blight of rice corresponding to each of the plots based on a correlation relationship and the rice leaf chlorophyll content and the rice plant WC corresponding to each of the plots, wherein the correlation relationship is a correlation relationship between the rice leaf chlorophyll content, the rice plant WC, and the incidence of the bacterial blight of rice;
screening characteristic variables of the rice spectral reflectance based on a score of a variable importance in the projection (VIP) index of the first regression model and a score of a VIP index of the second regression model to obtain a spectral index (SI); and
calculating a correlation between the SI and the incidence of the bacterial blight of rice, and generating a visual distribution map of the severity of the bacterial blight of rice in the study area based on the correlation between the SI and the incidence of the bacterial blight of rice.

2. The method for predicting a severity of bacterial blight of rice based on multi-phenotypic parameters according to claim 1, further comprising:
calculating a correlation between the SI and the rice leaf chlorophyll content, and generating a visual distribution map of the rice leaf chlorophyll content in the study area based on the correlation between the SI and the rice leaf chlorophyll content;
and/or, calculating a correlation between the SI and the rice plant WC, and generating a visual distribution map of the rice plant WC in the study area based on the correlation between the SI and the rice plant WC;
and/or, calculating the correlation between the SI and the rice leaf chlorophyll content, calculating the correlation between the SI and the rice plant WC, and generating a visual distribution map of the multi-phenotypic parameters of the rice in the study area based on the correlation between the SI and the incidence of the bacterial blight of rice, the correlation between the SI and the rice leaf chlorophyll content, and the correlation between the SI and the rice plant WC, wherein the multi-phenotypic parameters comprise the incidence of the bacterial blight of rice, the rice leaf chlorophyll content, and the rice plant WC.

3. The method for predicting a severity of bacterial blight of rice based on multi-phenotypic parameters according to claim 1, further comprising: constructing a sample database, wherein the sample database comprises the first sample data set, the second sample data set, and a third sample data set; and the third sample data set comprises a plurality of incidences of bacterial blight of the sample rice and a leaf chlorophyll content and a plant WC of the sample rice corresponding to each of the incidences of the bacterial blight of the sample rice.

4. The method for predicting a severity of bacterial blight of rice based on multi-phenotypic parameters according to claim 3, wherein a process of determining the leaf chlorophyll content of the sample rice is as follows:
determining the leaf chlorophyll content of the sample rice corresponding to each of the plots in the sample area using a soil-plant analysis development (SPAD)-502 chlorophyll meter.

5. The method for predicting a severity of bacterial blight of rice based on multi-phenotypic parameters according to claim 3, wherein a process of determining the plant WC of the sample rice is as follows:
calculating the plant WC of the sample rice corresponding to each of the plots in the sample area using a wet basis WC method.

6. The method for predicting a severity of bacterial blight of rice based on multi-phenotypic parameters according to claim 3, wherein a process of determining the first regression model is as follows:
determining the first regression model according to a partial least square regression (PLSR) method and the first sample data set.

7. The method for predicting a severity of bacterial blight of rice based on multi-phenotypic parameters according to claim 3, wherein a process of determining the second regression model is as follows:
determining the second regression model according to a PLSR method and the second sample data set.

8. The method for predicting a severity of bacterial blight of rice based on multi-phenotypic parameters according to claim 1, wherein a process of determining the first regression model is as follows:
determining the first regression model according to a partial least square regression (PLSR) method and the first sample data set.

9. The method for predicting a severity of bacterial blight of rice based on multi-phenotypic parameters according to claim 1, wherein a process of determining the second regression model is as follows:
determining the second regression model according to a PLSR method and the second sample data set.

10. The method for predicting a severity of bacterial blight of rice based on multi-phenotypic parameters according to claim 1, wherein a process of calculating a rice spectral reflectance corresponding to each of the plots based on the multi-spectral images specifically comprises:
pre-processing the multi-spectral images; and
extracting rice spectral reflectances from the pre-processed multi-spectral images using environment for visualizing images (ENVI) software, so as to determine the rice spectral reflectance corresponding to each of the plots.

11. A system for predicting a severity of bacterial blight of rice based on multi-phenotypic parameters, comprising:
a multi-spectral image obtaining module configured to obtain multi-spectral images of rice in a study area, wherein the study area comprises a plurality of plots;
a rice spectral reflectance calculation module configured to calculate a rice spectral reflectance corresponding to each of the plots based on the multi-spectral images;
a rice leaf chlorophyll content and rice plant WC calculation module configured to determine a rice leaf chlorophyll content corresponding to each of the plots based on the rice spectral reflectance and a first regression model, and determine a rice plant WC corresponding to each of the plots based on the rice spectral reflectance and a second regression model, wherein the first regression model is determined based on a first sample data set; the second regression model is determined based on a second sample data set; the first sample data set comprises a plurality of spectral reflectances of sample rice and a leaf chlorophyll content of the sample rice corresponding to each of the spectral reflectances of the sample rice, and the second sample data set comprises the plurality of spectral reflectances of the sample rice and a plant WC of the sample rice corresponding to each of the spectral reflectances of the sample rice; and the sample rice is rice under stress of the bacterial blight;
a rice bacterial blight incidence determination module configured to determine an incidence of the bacterial blight of rice corresponding to each of the plots based on a correlation relationship and the rice leaf chlorophyll content and the rice plant WC corresponding to each of the plots, wherein the correlation relationship is a correlation relationship between the rice leaf chlorophyll content, the rice plant WC, and the incidence of the bacterial blight of rice;
an SI determination module configured to screen characteristic variables of the rice spectral reflectance based on a score of a VIP index of the first regression model and a score of a VIP index of the second regression model to obtain an SI; and
a visual distribution map generation module for the severity of the bacterial blight of rice configured to calculate a correlation between the SI and the incidence of the bacterial blight of rice, and generate a visual distribution map of the severity of the bacterial blight of rice in the study area based on the correlation between the SI and the incidence of the bacterial blight of rice.

12. The system for predicting a severity of bacterial blight of rice based on multi-phenotypic parameters according to claim 11, further comprising:
a visual distribution map generation module for the rice leaf chlorophyll content configured to calculate a correlation between the SI and the rice leaf chlorophyll content, and generate a visual distribution map of the rice leaf chlorophyll content in the study area based on the correlation between the SI and the rice leaf chlorophyll content;
and/or, a visual distribution map generation module for the rice plant WC configured to calculate a correlation between the SI and the rice plant WC, and generate a visual distribution map of the rice plant WC in the study area based on the correlation between the SI and the rice plant WC;
and/or, a visual distribution map generation module for the multi-phenotypic parameters of the rice configured to calculate the correlation between the SI and the rice leaf chlorophyll content, calculate the correlation between the SI and the rice plant WC, and generate a visual distribution map of the multi-phenotypic parameters of the rice in the study area based on the correlation between the SI and the incidence of the bacterial blight of rice, the correlation between the SI and the rice leaf chlorophyll content, and the correlation between the SI and the rice plant WC, wherein the multi-phenotypic parameters comprise the incidence of the bacterial blight of rice, the rice leaf chlorophyll content, and the rice plant WC.
